(19) United States
(12) Patent Application Publication
(10) Pub. No.: US 2010/0312314 A1
(43) Pub. Date: Dec. 9, 2010

(54) Apparatus for Increasing Blood Perfusion and Improving Heat Sinking to Skin

(75) Inventors: Donald A. Ice, Milpitas, CA (US); Rudolf J. Hofmeister, San Jose, CA (US); Ueyn L. Block, Menlo Park, CA (US); Jan Lipson, Cupertino, CA (US)

Correspondence Address: FENWICK & WEST LLP, SILICON VALLEY CENTER, 801 CALIFORNIA STREET, MOUNTAIN VIEW, CA, 94041, US

(73) Assignee: C8 MEDISENSORS INC., Los Gatos, CA (US)

(21) Appl. No.: 12/786,699

(22) Filed: May 25, 2010

Related U.S. Application Data
(60) Provisional application No. 61/184,056, filed on Jun. 4, 2009.

(51) Int. Cl. A61F 7/00

(52) U.S. Cl. 607/100; 607/96

(57) ABSTRACT

An apparatus for increasing the blood perfusion in skin by elevating the temperature, and for providing superior heat sinking to the skin of thermally dissipative devices is disclosed. The increased perfusion gives rise to improved thermal transport properties near the site of elevated temperature which is advantageously used by thermally connecting the dissipative devices to the skin. The heat generated by the thermally dissipative devices can supplement or replace separate heating elements to elevate the skin temperature. Alternatively, thermal isolation of the heated area of the skin from the heat sinks of the dissipative devices can minimize the temperature of the skin in contact with the heat sinks.

### CROSS-REFERENCE TO RELATED APPLICATION

This application claims priority under 35 U.S.C. §119(e) to U.S. Provisional Patent Application Ser. No. 61/184,056, filed Jun. 4, 2009, entitled “Apparatus For Increasing Blood Perfusion And Improving Heat Sinking To Skin,” which is incorporated herein by reference in its entirety.

### BACKGROUND OF THE INVENTION

1. The Field of the Invention

This invention relates to improving the heat-sinking properties of living biological tissue through which blood is passing by controlling the temperature, which in turn affects local blood perfusion.

2. Background and Relevant Art

In some applications, one or more properties of living tissue are measured by mounting some device or ensemble of devices onto the tissue, wherein some components, which the device comprises, dissipate heat. If it is necessary to heat sink the thermally dissipative components, it may not be advantageous to do so using free convection of air, because the heat sinking capacity may not be adequate. It also may not be advantageous to use forced air convection because of increased power consumption, which is particularly a concern for battery-operated devices. In some circumstances, the biological tissue must be relied upon to provide an advantageous heat sink for heat dissipating components.

The thermal conductivity of skin is low unless it is well-perfused with blood, hence it may not be suitable for heat sinking unless perfusion is adequate at the heat sink site.

Also, in some applications where a property of the biological tissue is being measured, it is advantageous to control the perfusion of blood within the tissue for purposes of measurement accuracy. An example of such a case is the measurement of glucose in human skin, wherein adequate blood perfusion is necessary for the local concentration of glucose in blood and interstitial fluid to reach equilibrium with the average glucose concentration in the body's total blood volume.

In addition, in applications where low dark current optical detectors are required, it is advantageous to maintain the temperature of the detectors at a value as low as possible consistent with power consumption limitations for cooling. Thus, the sink temperature for the detector or for a cooler for the detector, if one is employed, should be as low as possible, whereas for example, the temperature of the biological sample in the neighborhood of the measurement may advantageously be higher in order to increase blood perfusion. Hence, it is useful to have some means of providing a temperature difference between the heat sinks for the different heat-dissipating components and for the measurement site on the biological sample.

Blood perfusion can rise by as much as one order of magnitude in a biological sample if it is heated from room temperature to the neighborhood of 40° C. as is demonstrated in “Effect of high local temperature on reflex cutaneous vasodilation,” W. F. Taylor et. al., J. Appl. Physiol.: Respirat. Environ. Exercise Physiol. 57 (1): 191-196, 1984. In U.S. Pat. No. 7,509,153, Blank et al. discloses an apparatus for controlling skin perfusion wherein the temperature of the site at which glucose is measured is controlled. The above patent is a continuation-in-part of U.S. Pat. Nos. 6,640,117 and 7,039,446. Blank teaches a means of controlling perfusion of blood in skin by control of temperature and of monitoring the effects thereof spectroscopically, in which monitoring and temperature control can be parts of a closed loop system. Consideration is not given to heat sinking to the skin, of heat dissipative components that may be part of the apparatus or of maintaining temperature differentials between the component-heated skin temperatures and the temperature of the measurement site. Neither is there an arrangement that assures adequate blood perfusion in the neighborhood of the heat sinks for thermally dissipative components.

### BRIEF SUMMARY OF THE INVENTION

These and other limitations are addressed by the present invention, which discloses an apparatus whereby the thermal conduction of the heat sinks for the thermally dissipative components which are on the surface of the biological sample can be enhanced by increasing local blood perfusion. In addition, it is possible to maintain a different temperature for the component heat sinks and for the site at which a property of the biological tissue is measured. From the requirement of continuity on fluid flow it can be seen that perfusion must be increased in areas neighboring the heated site, thereby improving thermal conduction at these neighboring locations while not increasing their temperature to the same degree as that of the heated site. It is shown how thermal isolation between the different regions can be maintained, where the apparatus makes contact with the biological sample. It is also shown how this thermal isolation can be beneficially used to isolate a heat sink associated with an optical detector from that associated with an optical source, both of which are thermally isolated from the site at which a property of the biological sample is measured.

### BRIEF DESCRIPTION OF THE DRAWINGS

FIG. 1 is a block diagram of an apparatus which can maintain different temperatures of the biological sample at different sites, and which is used to measure a property of the biological sample at one or more sites, in accordance with an embodiment of the invention.

FIG. 2 is a block diagram of an apparatus with the functions of FIG. 1 that is specific to making an optical measurement of a property of the biological sample, in accordance with an embodiment of the invention.

FIG. 3 is an isometric drawing of a preferred embodiment of an apparatus according to the functions described with reference to FIG. 2.

### DETAILED DESCRIPTION OF THE PREFERRED EMBODIMENTS

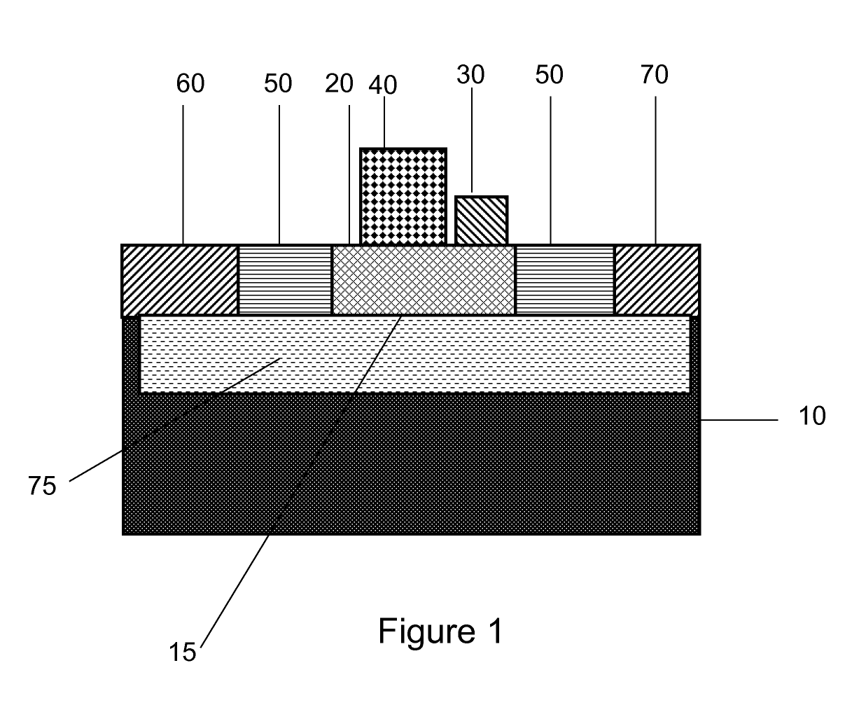

FIG. 1 illustrates a biological sample 10 through which blood is passing, and for which the flow of blood is an increasing function of temperature over some temperature range. A heater 20 is used to heat that portion of the biological sample 10 for which a property is to be measured, such as the glucose concentration in human blood. A device 30 monitors the temperature of the measurement site 15 which is in thermal equilibrium with the heater 20. A measurement apparatus 40 is used to measure a property of the measurement site 15 of the biological sample 10. Thermal insulation 50 impedes the flow of heat from the heater 20 to other areas of the biological sample 10. A heat sink 60 for one heat dissipating component is contained in the measurement apparatus 40. A second heat sink 70 for a second heat dissipating component is also contained in the measurement apparatus 40.

When the temperature at the measurement site 15 is increased, the flux of blood into and out of the biological tissue 10 in the neighborhood of the measurement site 15 is increased. In one example, biological tissue is heated in an area that is at least 20 mm². The increased flux of blood persists to some distance from the heated area, and the zone of substantially increased flux 75 is shown in FIG. 1 as part of the biological sample 10. The local thermal conduction of the sample 10 can be affected greatly by the blood flow when the sample is composed of biological tissue whose thermal conductivity is low when the temperature is not elevated above the typical equilibrium temperature for the sample with no heat applied. Such is the case for human skin. If it is necessary to heat-sink a thermally dissipative component with low thermal impedance over a limited area, the increased blood flow can be critical. If heat sinks are connected to the region of substantially increased blood flow 75, the thermal impedance of the heat path from a thermally dissipative device can be minimized.

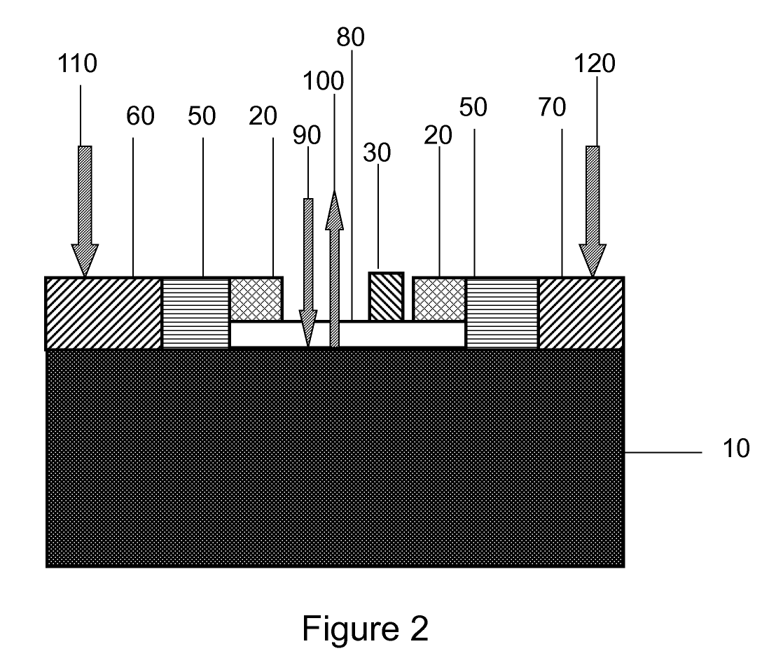

FIG. 2 is a block diagram which shows the basic implementation of the scheme of FIG. 1 to a device that performs an optical measurement on the biological sample 10. An optical window 80 has been interposed between the heater 20 and the sample 10. Light 90 can be injected into the sample 10 through the window 80 and scattered light 100 can pass through the window 80 as well. Heat sink 60 is connected to the heat flow 110 from the optical source. Heat sink 70 is connected to the heat flow 120 from the optical detector. It is understood that different or additional heat dissipative devices can be connected to heat sinks 60 and/or 70 or to other heat sinks. The embodiment shown in FIG. 2 is particularly advantageous when the heat flow from the optical source is large, but it is desired to maintain the detector at minimum temperature, as the two heat sinks 60 and 70 are thermally isolated, yet each is improved in thermal impedance because of the heating in the neighborhood.

The heater 20 is suitably chosen to be a resistive heater, which in a particularly preferred embodiment can be fabricated in a flex circuit using a nickel-chromium resistance conductor, or a conductor from some other resistive alloy. Temperature sensor 30 can be chosen to be a thermistor or a thermocouple, for example. In one embodiment, the optical window 80 should be chosen from a material whose thermal conductivity is much greater than that of skin, to assure a uniform temperature distribution at the measurement site 15. Good choices are silicon-carbide single-crystal material, sapphire, or diamond for visible or near-infrared radiation. Low-doped silicon is an excellent choice for radiation in the 1-6 um wavelength region.

The measurement apparatus, if optical, can be suitable for Raman spectroscopy, near-infrared spectroscopy, mid-infrared spectroscopy, optical coherence tomography, and diffuse reflectance measurement, but is not limited to these applications.

Properties that can be measured include but are not limited to the concentration of an analyte such as glucose, hemoglobin, water, triglycerides, or electrolytes. Additionally properties such as temperature, pulse rate, and blood perfusion can be included.

The insulator 50 can be chosen to be a polymer or air. Silica aerogel is also a good choice to lessen the heat transport by convection.

Heat sinks 60 and 70 are suitably chosen from the high thermal conductivity metals such as aluminum or copper.

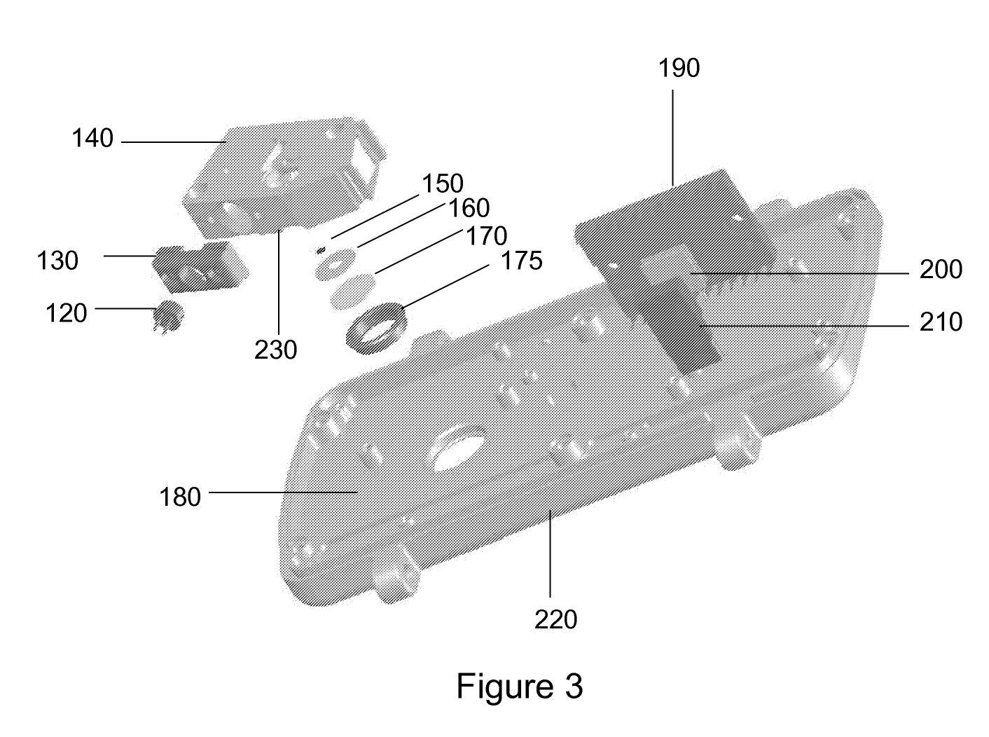

FIG. 3 is an isometric exploded view of a particular preferred embodiment, which is suitable for use on the skin of living mammalian organisms. A laser 120 is mounted by laser mount 130 to a block 140, to which a thermistor 150, a heater on flex-circuit 160, a window 170, and a window retainer 175 are also mounted. The bottom surface 230 of the block 140 rests against the top surface 180 of the base 220. A packaged detector 190, a thermoelectric cooler 200, and a heat sink 210 are also mounted to the base 220, as shown, for example, in FIG. 3, to make a transition from the hot side of the cooler 200 to the base, 220.

The heater 160 heats the window 170 which is advantageously fabricated in silicon carbide, sapphire, or diamond for visible and near-infrared applications, in one embodiment. The window retainer 175 is fabricated from a low thermal conductivity material, such as a polymer, and thermally isolates the heated window 170 from the base 220 assuring that the heat is applied only in the area desired. The base 220 is in contact with the skin on the side opposite of top side 180. The base 220 is advantageously fabricated in aluminum to achieve good thermal conductivity. The thermoelectric cooler 200 cools detector 190 and the heat from its hot side is deposited in heat sink 210 and then flows into the base 220. The location of the heat sink 210 is well-spaced away both from heated window 170 and the assembly containing the laser 120 such that the skin is not elevated in temperature either by the heat flow from heated window 170 or the heat dissipated by the laser 120. This allows the thermoelectric cooler 200 to achieve a lower temperature on its cold side for fixed power consumption because the temperature of the heat sink 210 to the hot side of the cooler 200 is minimized. If cooling is not required, the thermoelectric cooler 200 can be omitted and the detector 190 can be mounted directly to heat sink 210.

Blood perfusion will be high if skin temperature of about 40° C. is maintained in the neighborhood of the heated window 170 which is in contact with the skin. In some embodiments, the portion of the biological tissue that is heated has a temperature greater than 20° C. and less than 50° C. The block 140 conducts heat from the laser 120 to the base 220 in the neighborhood of the heated window 170 where blood perfusion is still high, but the temperature is not as highly elevated as at the heated window 170 because of the insulation provided by the window retainer 175. This arrangement provides both low thermal impedance and a lower heat sink temperature for the laser 120.

In one embodiment, FIG. 3 is approximately to scale and the window 170 is in contact with the skin over a diameter of about 8 mm. This has been found to be a sufficient area to obtain locally the maximum available increase in blood perfusion in human skin.

In another preferred embodiment, window retainer 175 can be made with high thermal conductivity, for example, exceeding 40 W/m° K. The heat sink for the laser 120 and the heated window 170 would then be thermally connected. This arrangement can be advantageous when the blood perfusion is required to be increased for other reasons besides improving heat sinking and it is desired to do so by heating with minimum power consumption. The proposed arrangement would then utilize the heat generated by the laser 120 to heat the skin by means of thermal conduction through items 130, 140, and 220, allowing reduced heating from the heater 160 and lower net power consumption.

Although the detailed description contains many specifics, these should not be construed as limiting the scope of the invention, but merely as illustrating different examples and aspects of the invention. It should be appreciated that the scope of the invention includes other embodiments not discussed in detail above. Various other modifications, changes and variations which will be apparent to those skilled in the art may be made in the arrangement, and details of the apparatus of the invention disclosed herein without departing from the spirit and scope of the invention.

1. An apparatus for improving heat sinking of a thermally dissipative component to biological tissue comprising:
a heat sink connected to the thermally dissipative component and connected to the biological tissue at a first location, and
a means for heating a portion of the biological tissue at a position distinct from the first location so as to increase blood perfusion in a region of the biological tissue which includes the first location.

2. The apparatus of claim 1, further comprising thermal insulation between the heat sink and the means for heating.

3. The apparatus of claim 1, wherein the biological tissue is skin.

4. The apparatus of claim 1, wherein the portion of the biological tissue that is heated has a temperature greater than 20° C. and less than 50° C.

5. The apparatus of claim 1, wherein the thermally dissipative component is an optical emitter.

6. The apparatus of claim 1, wherein the thermally dissipative component is an optical detector.

7. The apparatus of claim 1, wherein the thermally dissipative component is a thermoelectric cooler.

8. The apparatus of claim 1, wherein the means for heating comprises a resistive heater.

9. The apparatus of claim 1, wherein the means for heating comprises an optical emitter.

10. The apparatus of claim 1, wherein the means for heating is attached to an optical window which is in contact with the biological tissue.

11. The apparatus of claim 10, wherein the window has a thermal conductivity that exceeds 40 W/m° K.

12. The apparatus of claim 1, wherein the means for heating a portion of biological tissue heats an area that is at least 20 mm².
